(19) United States
(12) Patent Application Publication
(10) Pub. No.: US 2005/0177110 A1
(43) Pub. Date: Aug. 11, 2005

(54) Device for locking syringes to infusion pumps

(76) Inventor: Graziano Azzolini, Cavezzo (IT)

Correspondence Address: MODIANO & ASSOCIATI, Via Meravigli, 16, MILANO, 20123, IT

(21) Appl. No.: 11/050,837

(22) Filed: Feb. 7, 2005

(30) Foreign Application Priority Data
Feb. 6, 2004 (IT) MO2004A000027

(51) Int. Cl. A61M 37/00

(52) U.S. Cl. 604/151

(57) ABSTRACT

A device for locking syringes to infusion pumps comprising elements for supporting a syringe, of the type that comprises a substantially cylindrical body inside which a plunger is inserted so that it can slide axially, the supporting element being rigidly associable with the substantially cylindrical body, and a coupling element for temporarily coupling the supporting element to an anchoring element of a pump.

The present invention relates to a device for locking syringes to infusion pumps.

### BACKGROUND OF THE INVENTION

Infusion pumps for syringes are known which are used in the therapeutic or diagnostic field for the controlled and automated infusion of drugs, contrast media or other fluids.

Known pumps are substantially constituted by a supporting frame, with which means for locking the cylindrical body of a syringe and means of the linear automated type for actuating the sliding of the plunger of the syringe are associated.

An electronic circuit controls and actuates the actuation means in order to dispense the fluid contained in the syringe according to an infusion program (time, flow-rate, speed et cetera) that can be set from the outside by an operator by means of a keypad and a display.

Conventional locking means comprise a block, which is rigidly coupled to the pump frame and in which there is a slot in which it is possible to insert the wings formed at the end of the cylindrical body from which the plunger protrudes, and a clamp, which wraps around the cylindrical body in an upper region; the block prevents the axial sliding of the cylindrical body and the clamp prevents its rotation.

These conventional locking means are not free from drawbacks, including the fact that they are structurally and constructively very complicated, scarcely versatile and adaptable, and in particular do not allow oscillation of the syringe.

Other drawbacks of known locking means consist in that they cause operations for coupling and uncoupling the syringes to be awkward to perform on the part of health workers and in that they cannot be used and adapted easily to lock syringes to the rotating shafts with which certain infusion pumps are provided in order to make said syringes oscillate so as to mix the fluid contained therein.

### SUMMARY OF THE INVENTION

The aim of the present invention is to eliminate the drawbacks noted above of known locking means, by providing a device for locking syringes to infusion pumps that is structurally and constructively simple, is versatile in use and adaptable to the various dimensions of the syringes that can be used, allows to facilitate and simplify the operations performed by health workers to couple and disengage the syringes, and can be used easily to fix the syringes to rotating shafts with which the infusion pumps can be provided.

Within this aim, an object of the present invention is to provide a device that is simple, relatively easy to provide in practice, safe in use, effective in operation, and has a relatively low cost.

This aim and this and other objects that will become better apparent hereinafter are achieved by the present device for locking syringes to infusion pumps, characterized in that it comprises means for supporting a syringe, of the type that comprises a substantially cylindrical body inside which a plunger is inserted so that it can slide axially, said means being rigidly associable with said substantially cylindrical body, and means for temporarily coupling said supporting means to an anchoring element of said pump.

### BRIEF DESCRIPTION OF THE DRAWINGS

Further characteristics and advantages of the present invention will become better apparent from the following detailed description of a preferred but not exclusive embodiment of a device for locking syringes to infusion pumps, illustrated by way of non-limiting example in the accompanying drawings, wherein:

FIG. 1 is an exploded schematic perspective view of a device for locking syringes to infusion pumps according to the invention;

FIG. 2 is a schematic perspective view of the device of FIG. 1, applied to an infusion pump;

FIG. 3 is a schematic top plan view of the device of FIG. 2.

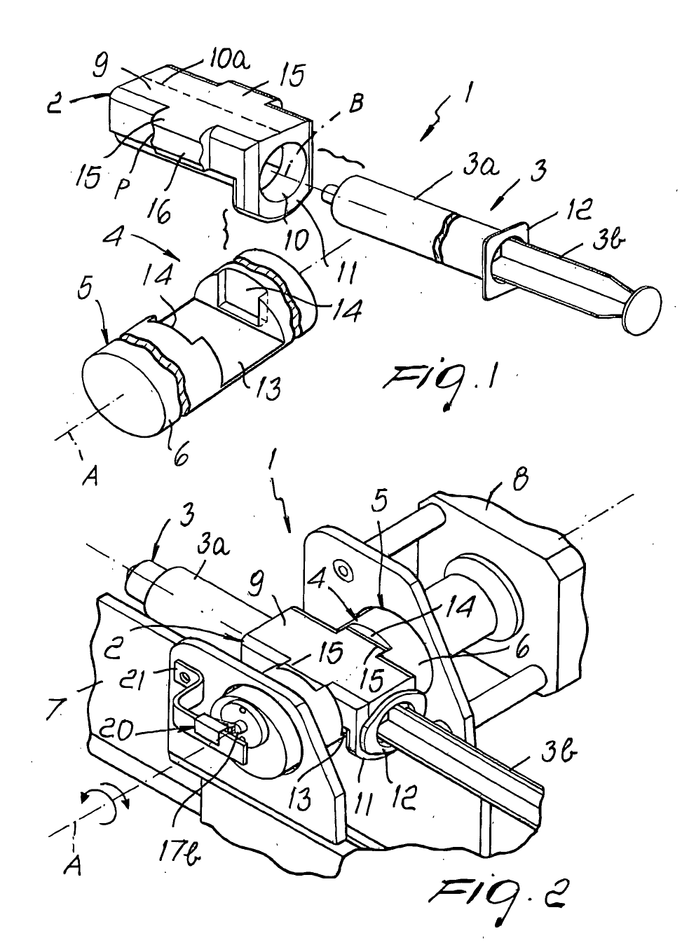

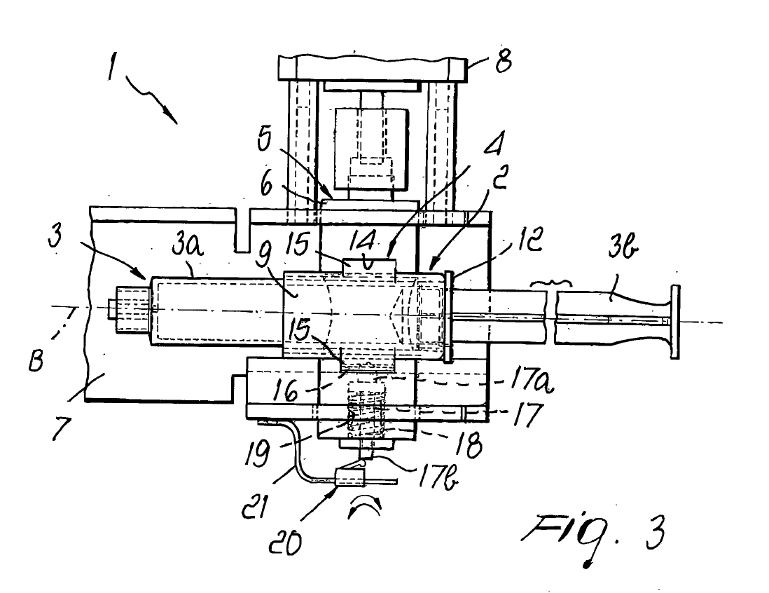

### DESCRIPTION OF THE PREFERRED EMBODIMENTS

With reference to the figures, the reference numeral 1 generally designates a device for locking syringes to infusion pumps, which are generally not shown as they are of a known type.

The device 1 comprises supporting means 2 for supporting a syringe 3, of the type that comprises a substantially cylindrical body 3a and inside which a plunger 3b is inserted so that it can slide axially, said supporting means being rigidly associable with the body 3a, and coupling means 4 for temporarily coupling the supporting means 2 to an anchoring element 5 formed in a pump.

The anchoring element 5 can be constituted for example by a shaft 6, which is provided with a longitudinal axis A that is substantially perpendicular to a longitudinal axis B of the syringe 3, is arranged substantially horizontally, and is supported so that it can rotate about its own longitudinal axis A by a frame 7 of the pump.

The shaft 6 is associated with motor means 8, which are suitable to rotate it alternately through a preset angle; the syringe 3, which is anchored to the shaft 6, is thus made to oscillate in order to mix the components of the fluid to be infused that are contained therein.

The supporting means 2 comprise a substantially tubular element 9, in an internal cavity 10 of which it is possible to insert the body 3a, fixing means for example of the interlocking, adhesive or interference-coupling type for fixing the element 9 to the body 3a being provided.

In one possible embodiment, the fixing means comprise a longitudinal slit 10a that is formed in the element 9, the inside diameter of which, in the configuration for disengagement from the body 3a, is substantially smaller than the outside diameter of the body 3a.

A substantially annular surface 11 for the abutment of wings or flange 12, formed proximate to the end of the body 3a in which the plunger 3b is inserted, is formed at one end of the element 9.

The coupling means 4 can be of the interlocking and/or barrier and/or friction types.

In the illustrated embodiment, the coupling means 4 comprise a receptacle 13 for containing the supporting means 2, which is formed in the anchoring element 5 and has a profile, i.e. a peripheral shape, that substantially duplicates or corresponds to the profile, i.e. the peripheral shape, of a corresponding portion of the outer lateral surface of the supporting means 2.

Cavities 14 are formed in the receptacle 13, and corresponding protrusions 15 formed in the supporting means 2 can be inserted therein; the cavities 14 and the protrusions 15 are prism-shaped.

Moreover, the supporting means 2 comprise a portion 16, which has a contoured profile P and is suitable to mate with a likewise contoured head 17a of a stem 17 that is inserted so that it can slide axially, with the interposition of elastic means 18, in a guide 19 that is formed in the anchoring element 5 at the receptacle 13; the head 17a protrudes within the receptacle 13 and the coupling between said head and the portion 16, together with the reaction of the elastic means 18, is suitable to fix the supporting means 2 to the anchoring element 5.

Means 20 for sensing presence of the syringe 3, such as on-off switches, sensors or the like, are further provided and are associated with the anchoring element 5; in particular, the sensing means 20 are fixed to a plate 21, which is anchored to the frame 7 proximate to an end of the anchoring element 5 at which there is an opening, from which the end 17b of the stem 17 that lies opposite the head 17a protrudes.

The stem 17 is suitable to activate the sensing means 20; by coupling the supporting means 2, to which the syringe 3 is fixed, to the cavity 13, their portion 16 mates with the head 17a of the stem 17, causing it to slide toward the outside of the anchoring element 5, therefore causing the activation of the sensing means 20, which report to a central control and monitoring unit the presence of the supporting means 2, i.e., of the syringe 3 that is rigidly coupled thereto.

In practice it has been found that the described invention achieves the intended aim and object.

The locking device according to the invention is in fact structurally and constructively simple and versatile and can be adapted to syringes of various sizes; it allows to facilitate the operations for engaging and disengaging the syringes that are performed by health workers and it can be used easily to fix syringes to the rotating shafts with which infusion pumps may be provided.

The invention thus conceived is susceptible of numerous modifications and variations, all of which are within the scope of the appended claims.

All the details may further be replaced with other technically equivalent ones.

In practice, the materials used, as well as the shapes and dimensions, may be any according to requirements without thereby abandoning the scope of the protection of the appended claims.

The disclosures in Italian Patent Application No. MO2004A000027 from which this application claims priority are incorporated herein by reference.

1. A device for locking a syringe of the type that comprises a substantially cylindrical body having a longitudinal axis inside which a plunger is inserted so as to be axially slidable, to an infusion pump, comprising: supporting means for supporting the syringe, said supporting means being rigidly associable with said substantially cylindrical body; an anchoring element that is fixable to a frame of the pump; and coupling means for temporarily coupling said supporting means to said anchoring element.

2. The device of claim 1, wherein said anchoring element is constituted by a shaft, which extends along a longitudinal axis thereof that lays substantially at right angles to the longitudinal axis of the syringe body, said shaft being supported so as to perform rotation about said longitudinal axis thereof by the frame of the pump.

3. The device of claim 2, wherein said shaft is adapted to perform rotation in alternated directions.

4. The device of claim 1, wherein said supporting means comprise a substantially tubular element, adapted to allow insertion of said substantially cylindrical body therein, and fixing means for rigidly fixing said substantially tubular element to said substantially cylindrical body.

5. The device of claim 4, wherein said fixing means are selectable among interlocking, adhesive-bonding or interference-coupling fixing means.

6. The device of claim 4, wherein said fixing means comprise a longitudinal slit, which is formed in said substantially tubular element, said substantially tubular element having an inside diameter that is substantially smaller than an outside diameter of said substantially cylindrical body.

7. The device of claim 4, wherein said substantially cylindrical body is provided at an end thereof with protruding wings, said substantially tubular element comprising, at an end thereof in which the plunger is inserted, a substantially annular surface for the abutment of said wings.

8. The device of claim 1, wherein said temporary coupling means are selectable among interlocking and/or barrier and/or friction coupling means.

9. The device of claim 8, wherein said temporary coupling means comprises a receptacle for containing said supporting means, said receptacle being formed in said anchoring element.

10. The device of claim 9, wherein said containment receptacle has a peripheral shape that substantially duplicates a peripheral shape of a corresponding portion of an outer lateral surface of said supporting means.

11. The device of claim 10, wherein said supporting means comprise protrusions, said containment receptacle being provided with cavities that are shaped correspondingly to said protrusions, whereby the protrusions are insertable in said cavities.

12. The device of claim 11, wherein said cavities and said protrusions are prism-shaped.

13. The device of claim 9, wherein said anchoring element comprises a guide formed at said containment receptacle, and said supporting means comprises a portion that has a contoured profile, elastic means and a stem having a head, said contoured profile being adapted to mate with the head of the stem that is inserted axially slidable, with the interposition of said elastic means, in said guide, said head protruding within said containment receptacle, and mating coupling between said head and said portion having a contoured profile being suitable to fix said supporting means to said anchoring element.

14. The device of claim 13, comprising sensing means for sensing presence of said syringe which are associated with said anchoring element, said sensing means being of a type selectable among on/off switches, or sensors.

15. The device of claim 14, wherein said stem has an end that lies opposite to said head and protrudes externally from said anchoring element, said sensing means being arranged proximate to said end, and said stem being adapted to actuate said sensing means.
